(19) United States
(12) Patent Application Publication
(10) Pub. No.: US 2002/0045600 A1
(43) Pub. Date: Apr. 18, 2002

(54) Topical skin treatment for sea lice, insect bites and skin irritation

(76) Inventor: Laura M. Schwarzman, Boca Raton, FL (US)

Correspondence Address: John C Smith Esquire, Suite A 207, 4800 North Federal Highway, Boca Raton, FL, 33431, US

(21) Appl. No.: 09/611,400

(22) Filed: Jul. 7, 2000

Related U.S. Application Data
(60) Provisional application No. 60/142,840, filed on Jul. 8, 1999.

(51) Int. Cl. A61K 31/665; A01N 57/00

(52) U.S. Cl. 514/100

(57) ABSTRACT

A topical treatment for skin irritation that uses a spray-on liquid or a rub-on lotion based papain skin treatment for relief of symptoms related to skin irritation. Papain is suspended in a water solution that is spread on the surface of the affected area. The liquid solution ensures that the active ingredient contacts the skin without missing spots as may occur when using dry or powdered skin treatments. The papain is an active ingredient extracted from papaya which relieves the skin irritation caused by sea lice, insect bites, etc. The water solution is used to suspend the papain such that it more completely contacts the affected area, and further to maintain the papain in a fresh and active state. Optionally, aloe vera is included in the solution to increase the soothing effect on the skin in combination with the papain. In another preferred embodiment, citric acid and sodium benzoate are also used to increase shelf life.

### CROSS-REFERENCE TO RELATED APPLICATIONS

This application is a continuation of the commonly owned copending application entitled “Topical Skin Treatment for Sea Lice, Insect Bites and Skin Irritation”, filed Jul. 8, 1999, bearing U.S. Ser. No. 60/142,840 and naming Laura M. Schwarzman, the named inventor herein, as sole inventor, the contents of which is specifically incorporated by reference herein in its entirety.

### BACKGROUND OF THE INVENTION

1. Technical Field

The present invention relates to topical skin treatments. In particular, it relates to liquid and lotion skin treatments that are spread on the surface of the skin to alleviate the symptoms of skin irritation caused by sea lice, insect bites, and plant irritants such as poison oak and poison ivy.

2. Background Art

The prior art has provided a variety of treatments for skin irritation. For insect bites, such as mosquito bites, treatments such as Calamine lotion, have been used for many years. Cortisone treatment has also been used to treat numerous skin problems, including insect bites.

In addition to insects, there are numerous other sources of skin irritation. For example, numerous plants produce substances that have a toxic or irritating effect on skin. Poison Ivy, poison oak, poison sumac, etc. are only a few examples of many types of plants which have an injurious effect on human skin.

Insects and plants that attack people on land are only one source of skin irritation. There are also many waterborne hazards faced by humans. For individuals who swim in warmer coastal waters, sea lice represent a particularly disagreeable hazard. Sea lice is a commonly used colloquial expression which describes jelly fish larvae. Sea lice lodge themselves under the skin and result in unsightly welts and extremely irritated and sensitive skin. Treatment for sea lice is typically an agent designed to kill the cause of the irritation. The most common remedy being the use of chemicals such as ammonia to kill the offending organisms. While this will eventually resolve the problem, it does not provide any immediate relief to those being bitten by sea lice. Further, the use of ammonia exacerbates the stinging and burning sensation caused by the sea lice and actually increases user discomfort. It would be desirable to have a way to treat skin irritations caused by a variety of environmental conditions, including those conditions caused by waterborne nuisances such as sea lice, and land borne nuisances such as mosquitos, insects, and plants with substances that irritate skin (for example, poison ivy), which would immediately alleviate the pain and discomfort rather than increase it as the prior art treatments such as ammonia have done.

A disadvantage associated with treatments that do not alleviate pain and discomfort is that they create a situation in which the individual scratches the affected area, which further irritates the skin, and prolongs the healing process.

While addressing the basic desirability of treating sea lice to ensure their elimination, the prior art has failed to provide a method of treating skin irritations caused by sea lice, insect bites, etc. and which results in immediate relief of the symptoms.

### SUMMARY OF THE INVENTION

The present invention solves the foregoing problems by providing a topical treatment for skin irritation that use a spray, a liquid, or a lotion based papain skin treatment for skin irritation. Papain is suspended in an aqueous solution that is applied to the surface of the affected area. The liquid solution ensures that the active ingredient contacts the skin without missing spots as may occur when using dry or powdered skin treatments. The papain is an active ingredient extracted from papaya which relieves the skin irritation caused by sea lice, insect bites, etc. The water solution is used to suspend the papain such that it more completely contacts the affected area. Further, the aqueous solution maintains the papain in a fresh and active state. Optionally, aloe vera is included in the solution to increase the soothing effect on the skin in combination with the papain. In another preferred embodiment, citric acid and sodium benzoate are also used to increase shelf life of the aqueous papain solution.

### BRIEF DESCRIPTION OF THE DRAWINGS

FIG. 1 is a diagram that illustrates a prior art paste or powdered treatment.

FIG. 2 illustrates the liquid treatment which more completely covers the affected area.

FIG. 3 illustrates application of the liquid treatment with a spray bottle.

FIG. 4 illustrates the use of a reusable path to apply liquid treatment to the user's skin.

FIG. 5 illustrates a method of packaging a liquid solution of the pain with a disposable pad in a sealed package.

### DESCRIPTION OF THE PREFERRED EMBODIMENT

Prior to a detailed discussion of the figures, a general overview of the system will be presented. Presently, many people enjoy outdoor activities in which they come in contact with many natural hazards. For example, in beach areas there are many water born pests which can attack the skin of the user, or sting the user. Examples of these pests are sea lice, a common water born pest in the southeastern United States and in the Gulf of Mexico. When a swimmer is attacked by sea lice, the sea lice create large painful welts on the user's skin. These welts create substantial amounts of itching and pain. Likewise, jelly fish and other sea pest stings or bites may cause similar problems.

Likewise, on land, numerous insects, such as bees, spiders, mosquitoes, etc. can cause irritation and pain which is similar to the irritation and itching afflicted on swimmers by sea borne pests. In addition to insects, there many plants which carry toxic substances capable of the irritating the skin of the user. For example, poison ivy, poison oak, etc. will cause irritation and pain in the user's skin.

The preferred embodiment of the invention provides an aqueous solution of papain. The papain solution is preferably applied from a spray bottle. The user applies the papain skin treatment by spraying it on the user's skin. The advantage of using a liquid form of papain is that the liquid spray is uniformly applied to the user's skin and does not miss portions of the skin as would be the case if a solid papain formulation was used. When the papain is applied to the user's skin, it substantially reduces the swelling, irritation, and pain caused by the insects, sea pests, or plant toxins discussed above. The spray contains the protein known as papain, which is the proteolytic enzyme isolated from the latex of the green fruit and leaves of the Carica Papaya plant. Papain has been found to substantially reduce the swelling, and alleviate the itching and discomfort caused by damage to the skin by the irritants discussed above.

The invention uses a water-based formula because it allows the papain to be conveniently sprayed on the surface of the skin in a uniform manner. In addition, by suspending the papain in a water-based formulation, it has been found that the effectiveness of the papain is not only increased, but its shelf life is also increased. In contrast, solid forms of papain and to deteriorate rapidly, have a short shelf life, and are less effective because of the inability to adequately cover the skin surface of the user. To further increase its effective life span, some preservatives can be added to the aqueous solution. In particular, small amounts of preservatives such as citric acid and/or sodium benzoate can be added to the aqueous solution to increase shelf life. Typically, only a small amount of preservative would be added. For example, for a given volume of skin lotion, an amount of sodium benzoate on the order of 1/10 of 1 percent by volume would be used. Those skilled in the art will recognize that alternative preservatives can be used so long as they do not impair the effectiveness of the papain or otherwise cause any irritation when applied to a user's skin.

An alternative embodiment adds an additional ingredient, aloe vera, to the formulation. The aloe vera soothes the skin and works in conjunction with the papain to more rapidly eliminate the symptoms caused by the skin irritants. The overall percentages of ingredients can vary. The amount of water must be sufficient so that the liquid or lotion created by the formulation can be smoothly and easily applied to the skin. Likewise, any other liquid base can be substituted for water providing that it does not interfere with the papain or the aloe vera.

As discussed above, the addition of a small amount of preservatives is also useful for extending the shelf life of the formulation. To that end, a suitable preservative such as sodium benzoate may be used in small amounts to preserve the formulation in the same manner as preservatives are used on food products.

Those skilled in the art will recognize that while the preferred embodiment uses a liquid form which is applied by a spray bottle, the papain can also be made in a variety of viscosities which will allow it to be used as a lotion which is rubbed onto the user's skin, etc. Any suitable material may be used in the place of, or in combination with water, to increase the viscosity of the liquid solution to suit the intended application procedure. While a spray bottle with more effectively use low viscosity solutions based on water, an ointment would preferably use a higher viscosity medium to facilitate the process of rubbing the ointment onto the user's skin. As can be seen, a variety of methods can be used to apply a liquid to a surface such as skin. The preferred embodiment uses a spray bottle which results in a uniform application of the solution for a given area. However, an aqueous solution can be spread by rubbing it on via the users fingers, it can be spread on by first applying it to a pad and then rubbing the pad on the skin of the user, it can be pre-impregnated in disposable cloths or pads in sealed packages which can be opened, used, and discarded, etc. Likewise, by making the aqueous solution more viscous, it can be spread on the user's skin in the same manner as a skin lotion.

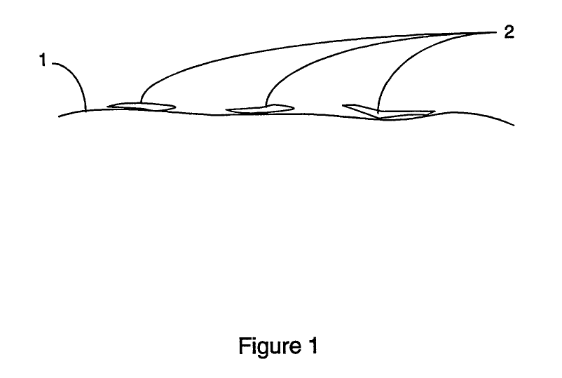

FIG. 1 is a prior art topical skin treatment which is based on a powder or paste formulation. In this figure, the surface of the individual's skin 1 has a powder or paste topical skin treatment 2 on it. As can be seen from this figure, the power or paste topical skin treatment 2 is not evenly applied and results in some areas of the skin 1 being treated and other areas of the skin 1 not being treated. As a result, the dry or semi dry topical skin treatments which are based on powder or paste formulations do not adequately cover the entire skin surface. Because powders and pastes do not adequately cover the entire skin surface, even if papain was used as part of the powder or paste, it would not be effectively applied to the skin surface areas which need treatment.

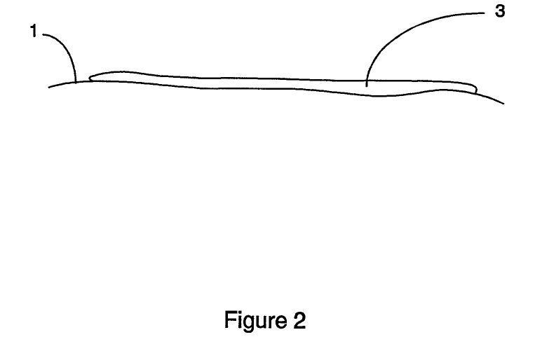

FIG. 2 illustrates the use of an aqueous papain solution 3 which is used as a liquid based topical skin treatment. As can be seen in this illustration, the aqueous papain solution 3 is applied to the entire skin surface 1 without leaving gaps. As a result of this more complete application, the skin 1 is more completely treated which in turn results in faster healing. When using a liquid papain solution 3, the solution can be formulated such that it varies in viscosity. By making the solution more viscous, the user may decide to spread the solution on affected skin areas with the users fmgers. However, by making the solution less viscous it is possible to use other devices such as spray bottles to apply the aqueous papain solution 3 to the user's skin.

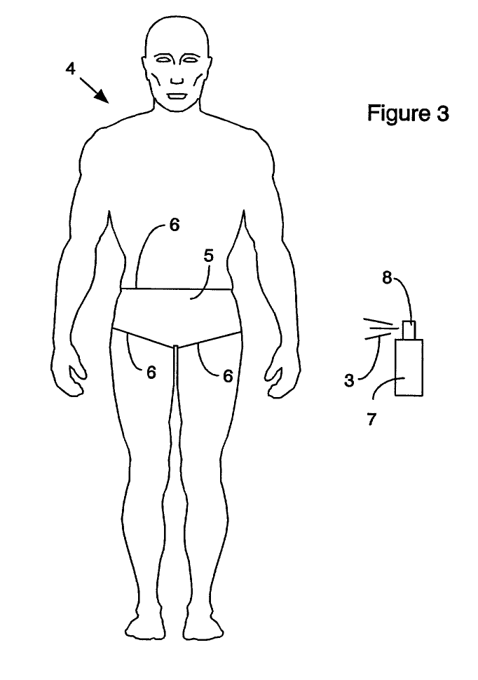

FIG. 3 illustrates the application of the liquid papain solution 3 to the skin of the user 4 with a spray bottle 7. The spray bottle may have a pump nozzle 8 or it may also use compressed gas to spray the liquid papain solution 3. The spray bottle 7 allows the user to easily spray affected areas. For certain pests, such as the lice, the affected areas would typically be found near the elastic seams 6 of a bathing suit 5.

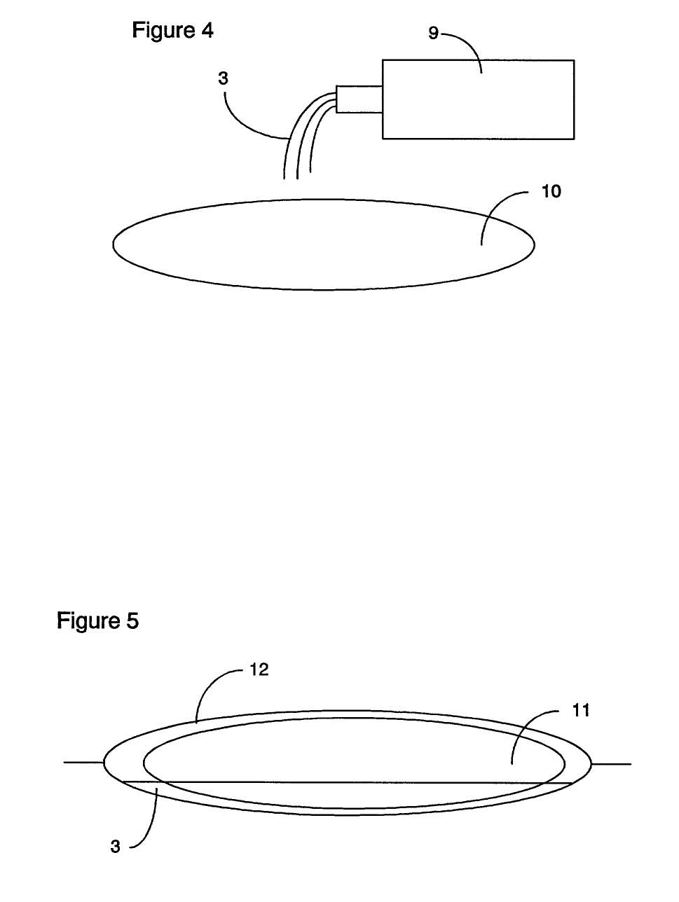

FIG. 4 a illustrates an alternative preferred embodiment in which a reusable pad 10 has an aqueous papain solution 3 applied to it from a supply bottle 9. The user 410 has the ability to apply the aqueous papain solution 3 to the user's skin by rubbing the reusable pad 10 on the affected areas.

FIG. 5 illustrates another preferred embodiment in which a self-contained application system includes a sealed package 12 that has within it a disposable pad 11 and a supply of aqueous papain solution 3. In this embodiment, the user would open the sealed package 12, remove the disposable pad 11 (which would have absorbed a substantial amount of the aqueous papain solution 3, rub the disposable pad 11 on the affected areas, and then discard the disposable pad 11 and the sealed package 12. This embodiment allows the user 4 to conveniently carry a small package which is available for use when needed.

An important advantage of all of the foregoing embodiments is that they use a liquid solution which provides a superior and more complete application of papain to injured areas of skin. Of course, those skilled in the art will recognize that a water-based aqueous solution is not necessary. Any suitable liquid base can be used so long as it does not interfere with the papain. Likewise, the use of other liquid's provides the user with the option of having different viscosities than that provided by ordinary water.

In addition to improving the completeness of the application of the skin treatment, the use of a liquid based formulation allows the individual to more easily apply the skin treatment without the use the special devices or applicators. This also results in reduced manufacturing costs since no application devices are needed other than the fingers of the user.

It has been found that the use of papain on irritated skin provides rapid and effective relief of symptoms such as pain, swelling, itching, etc. However, when papain is used for this purpose, it has also been found that it has a fmite shelf life. Papain in paste and/or powder forms is less effective due to that shorter shelf life. In addition, there is also the disadvantage discussed above in which skin treatments are not uniformly applied to the skin surface in paste or powder forms.

It has further been found that the shelf life and effectiveness of papain can be increased by creating a solution in which papain is applied to the user's skin 1 in liquid or lotion form. In the preferred embodiment, a water-based solution is used in which water and papain are mixed to form a liquid. In addition, optional ingredients to stabilize and preserve the formulation are added. The optional ingredients in the preferred embodiment are citric acid and a small amount (i.e. 0.1 percent) of the preservative sodium benzoate. In this liquid formulation, the papain will be evenly spread over the skin 1 when the lotion is applied by rubbing it on the skin 1.

Another advantage of the formulation used in the preferred embodiment is that it is safe to use, and in fact may even be ingested by the user without concern. Further, since all of the ingredients of the formula are natural products, the user does not have to be concerned about the use of non-natural chemicals.

In an alternative preferred embodiment, aloe vera is added to the formulation. The addition of aloe vera provides an additional skin soothing compound which further increases the relief of symptoms related to insect bites, sea lice bites, and other skin irritants such as poison ivy, poison oak, etc.

While the invention has been described with respect to a preferred embodiment thereof, it will be understood by those skilled in the art that various changes in detail may be made therein without departing from the spirit, scope, and teaching of the invention. For example, the formula can be implemented a liquid which is dispensed by a spray bottle, it can be implemented as a lotion which is rubbed on, etc. The percentages of ingredients can vary, etc.

I claim:

1. A liquid papain solution for use in treating skin conditions, comprising:
papain;
a liquid medium capable of carrying papain in suspended or dissolved form;
whereby the papain can be evenly distributed on a selected area of skin by distributing the liquid papain solution on that area.

2. A liquid papain solution, as in claim 1, wherein:
the liquid medium is water.

3. A liquid papain solution, as in claim 2, further comprising:
a preservative, the preservative dissolved in the liquid medium;
whereby the preservative extends the useful life of the liquid papain solution.

4. A liquid papain solution, as in claim 3, wherein:
the preservative is sodium benzoate.

5. A liquid papain solution, as in claim 2, further comprising:
aloe, the aloe suspended or dissolved in the liquid medium.

6. A liquid papain solution, comprising:
papain;
a liquid medium capable of carrying papain in suspended or dissolved form;
a spray bottle, the spray bottle having means to store a supply of the liquid papain solution and further having means to spray the liquid papain solution;
whereby the papain can be evenly distributed on a selected area of skin by spraying the liquid papain solution on that area.

7. A liquid papain solution, as in claim 2, wherein:
the liquid medium has a higher viscosity than the viscosity of water.

8. A liquid papain solution, as in claim 7, wherein:
the viscosity of the liquid medium is sufficient to allow the papain solution to be used as a lotion.

9. A liquid papain solution, as in claim 2, further comprising:
an applicator pad;
a storage container for holding a supply of liquid papain solution;
whereby the liquid papain solution can be poured on the applicator pad and applied to the user skin via the applicator pad.

10. A liquid papain solution, as in claim 2, further comprising:
a sealed package;
an applicator pad contained within the sealed package;
the liquid papain solution stored in the sealed package such that the applicator pad absorbs a substantial portion of the papain solution;
whereby the user can open the sealed package, remove the applicator pad, and apply the liquid papain solution by rubbing the applicator pad on a selected area of skin.

11. A method of applying liquid papain solution to selected areas of skin for use in treating skin conditions, including the steps of:
dissolving or suspending obtained in a liquid medium;
applying the liquid medium carrying the papain to a selected area of skin;
whereby the papain can be evenly distributed on a selected area of skin by distributing the liquid papain solution on that area.

12. A method, as in claim 11, including the additional step of:
using water as the liquid medium.

13. A method, as in claim 12, including the additional step of:
dissolving a preservative in the liquid medium;
whereby the preservative extends the useful life of the liquid papain solution.

14. A method, as in claim 13, including the additional step of:
using sodium benzoate as the preservative.

15. A method, as in claim 12, including the additional step of:
suspending or dissolving aloe in the liquid medium.

16. A method, as in claim 12, including the additional steps of:
using a spray bottle to store a supply of the liquid papain solution;
spraying, with the spray bottle, the liquid papain solution on a selected area of skin;
whereby the papain can be evenly distributed on a selected area of skin by spraying the liquid papain solution on that area.

17. A method, as in claim 12, including the additional step of:
using a liquid medium that has a higher viscosity than the viscosity of water.

18. A method, as in claim 17, including the additional step of:
increasing the viscosity of the liquid medium to allow the papain solution to be used as a lotion.

19. A method, as in claim 12, including the additional steps of:
storing a supply of liquid papain solution in a storage container;
dispensing a portion of the liquid papain solution from the storage container to an applicator pad;
wiping the applicator pad containing the liquid papain solution on a selected area of skin;
whereby the liquid papain solution can be poured on the applicator pad and applied to the user skin via the applicator pad.

20. A method, as in claim 20, including the additional steps of:
a sealed package;
sealing an applicator pad within a sealed package;
storing a supply of liquid papain solution in the sealed package such that the applicator pad absorbs a substantial portion of the papain solution;
whereby the user can open the sealed package, remove the applicator pad, and apply the liquid papain solution by rubbing the applicator pad on a selected area of skin.
